(19) United States
(12) Patent Application Publication
(10) Pub. No.: US 2010/0057113 A1
(43) Pub. Date: Mar. 4, 2010

(54) METHOD AND APPARATUS FOR INSERTION OF DEVICE IN TROCAR

(76) Inventors: Shalom Levin, Atlit (IL); Nir Altman, Kibbutz Kfar Etzion (IL); Arik Cohen, Zichron Ya'acov (IL); Guy Rogel, Haifa (IL)

Correspondence Address: David Klein;DEKEL PATENT LTD., Beit HaRof'im, 18 Menuha VeNahala Street, Room 27, REHOVOT, IL

(21) Appl. No.: 12/198,155

(22) Filed: Aug. 26, 2008

(51) Int. Cl. A61B 17/34; A61B 17/03

(52) U.S. Cl. 606/151; 128/898

(57) ABSTRACT

A method for insertion of a device in a trocar, including placing a finger trap over a reducible portion of a device that has a reducible perimeter, such that the finger trap applies a force on the reducible portion of the device to reduce its perimeter, and inserting the device into a trocar.

### FIELD OF THE INVENTION

The present invention generally relates to a method and an apparatus for insertion of devices, such as but not limited to, rolled meshes, in trocars or cannulas, such as in laparoscopic procedures.

### BACKGROUND OF THE INVENTION

Surgical trocars are used extensively in medical procedures, especially laparoscopic surgery. Trocars are used for inserting therethrough laparoscopic instruments, such as but not limited to, cutting tools, sutures, graspers, and the like, and as portals for devices, such as but not limited to a chest drain, intravenous cannula, and many more.

An example of trocar use is in hernia surgery. Hernias are abnormal protrusions of an organ (or organs) through a defect or natural opening in a covering membrane, muscle or bone. The defective gap or opening may be closed by sutures, and in cases of a larger defective gap, a mesh-sheet (or mesh, for short) may be applied over the gap. In some cases a mesh-sheet may be used for reinforcing a primary sutured defect in the abdominal wall. The mesh-sheet is fixed with sutures at peripheral edges thereof to the abdominal wall.

The mesh sheet is deployed and sutured in place by introducing the mesh sheet and tools through an incision into the body via a trocar or cannula. The mesh sheet may be in the form of a rolled sheet. For example, PCT published patent application WO 2004/080348 to the present assignee, the disclosure of which is incorporated herein by reference, describes apparatus that includes a rod formed with a handle portion and a coaxial roller portion. The mesh-sheet is wound around the roller portion. PCT patent application PCT/IL2008/000149 to the present assignee, the disclosure of which is incorporated herein by reference, improves upon the previous PCT application by providing a joint that increases the possibilities of maneuvering the roller portion of the device.

### SUMMARY OF THE INVENTION

The present invention seeks to provide a method and an apparatus for insertion of devices, such as but not limited to, rolled meshes, in trocars or cannulas, as is described more in detail hereinbelow.

The apparatus and method are particularly useful for the deployment and placement of a mesh-sheet in a body, e.g., the abdominal cavity or the inguinal space, such as for covering a hernial defect of a patient during a hernia repair in a laparoscopic procedure. The apparatus and method ensure the rolled mesh does not bunch or snag or unwind before and during insertion via the trocar or cannula.

There is thus provided in accordance with an embodiment of the present invention a method for insertion of a device in a trocar, including placing a finger trap over a reducible portion of a device that has a reducible perimeter, such that the finger trap applies a force on the reducible portion of the device to reduce its perimeter, and inserting the device into a trocar.

In accordance with one embodiment of the present invention, the method includes inserting the device into the trocar with the finger trap fitted over the device, and afterwards removing the finger trap from the device.

In accordance with an alternative embodiment of the present invention, the method includes first removing the finger trap from the device, and then inserting the device into the trocar.

In accordance with yet another embodiment of the present invention, the method includes leaving the finger trap on the reducible portion of the device for a sufficient time so that the force applied on the reducible portion of the device causes the reducible portion to take a set and remain with a reduced perimeter before insertion in the trocar.

In accordance with an embodiment of the present invention, a proximal end of the finger trap fits over a proximal portion of the device, a distal end of the finger trap fits over a distal portion of the device and a middle portion of the finger trap is enlarged and fits over the reducible portion of the device.

There is also provided in accordance with an embodiment of the present invention apparatus for insertion of a device in a trocar, including a finger trap fitted over a reducible portion of a device that has a reducible perimeter, such that the finger trap applies a force on the reducible portion of the device to reduce its perimeter.

### BRIEF DESCRIPTION OF THE DRAWINGS

The present invention will be understood and appreciated more fully from the following detailed description taken in conjunction with the drawings in which:

FIG. 1 is a simplified illustration of mesh deployment device of the prior art (PCT/IL2008/000149);

FIG. 2 is a simplified illustration of an apparatus for insertion of a device (e.g., the mesh of the mesh deployment device of FIG. 1) in a trocar, constructed and operative in accordance with an embodiment of the present invention;

FIG. 3 is a simplified illustration of the apparatus of FIG. 2 completely covering the mesh of the mesh deployment device of FIG. 1, just prior to insertion in a trocar;

FIG. 4 is a simplified illustration of the apparatus of FIG. 2 completely covering the mesh of FIG. 1, after insertion and pulling through the trocar; and

FIG. 5 is a simplified illustration of the mesh deployment device of FIG. 1, after insertion through the trocar and removal of the apparatus of FIG. 2, in accordance with an embodiment of the present invention.

### DETAILED DESCRIPTION OF EMBODIMENTS

The present invention seeks to provide a method and an apparatus for insertion of devices in trocars or cannulas. Throughout the description and claims, the term “trocar” will be used to encompass any tube-like instrument, such as but not limited to, trocars, cannulas and the like. The invention will be described for use with rolled meshes. However, the invention is not limited to rolled meshes and can be used for inserting any medical device, such as but not limited to, cutting tools, graspers, stents and the like through a trocar.

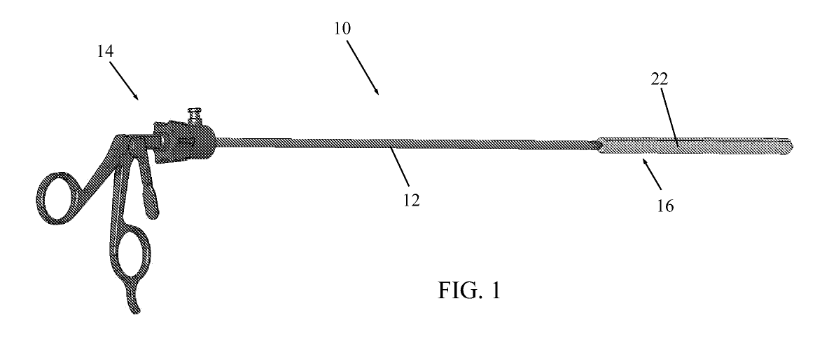

Reference is now made to FIG. 1, which illustrates mesh deployment device 10, which for exemplary purposes is that of PCT patent application PCT/IL2008/000149.

Briefly, mesh deployment device 10 includes a deployment rod 12 having a handle 14 at a proximal portion thereof and a roller portion 16 at a distal portion thereof. A mesh-sheet 22 (or simply mesh 22), which is made of a bio-compatible material as is well known in the art, is detachably attached to roller portion 16.

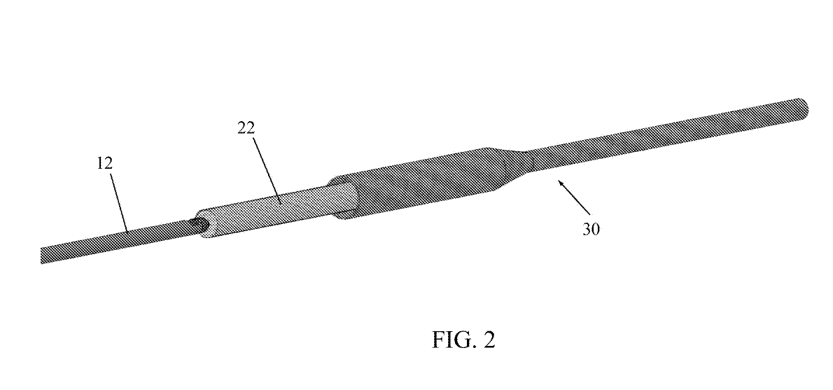

Reference is now made to FIG. 2, which illustrates an apparatus 30 for insertion of a device (e.g., the mesh deployment device 10 of FIG. 1) in a trocar, constructed and operative in accordance with an embodiment of the present invention.

Apparatus 30 is a finger trap (herein referred to as finger trap 30). Finger traps are available from a variety of manufacturers, such as Rycor Medical, Inc., 2053 Atwater Drive, North Port, Fla. 34288. Finger traps are typically constructed of cylindrical braids, wherein pulling the braid lengthens and narrows it. More specifically, the length of the braid increases as the angle between the woven threads and the diameter (and circumference) of the braid decreases. Thus, the more the finger trap is pulled the more the trap tightens on an object, because the circumference and diameter shrink as the trap tightens. Finger trap 30 may be made of any suitable medically safe material, such as but not limited to, fabric (any natural of artificial material used for sutures, for example) or metal (e.g., stainless steel). FIG. 2 illustrates pulling finger trap 30 over the distal end of mesh deployment device 10. It is noted that an end of finger trap 30 is manipulated to have an enlarged diameter for fitting over device 10. This is easily accomplished by pushing the end of finger trap 30 towards the middle of the trap, which enlarges the opening of that end of the trap.

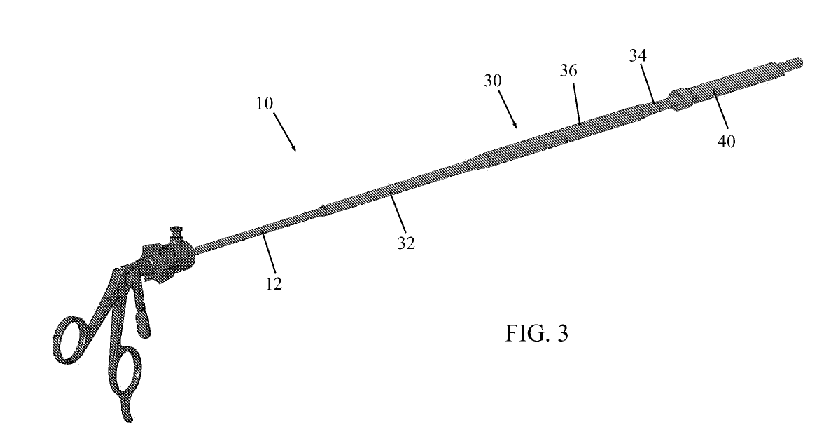

Reference is now made to FIG. 3, which illustrates finger trap 30 completely covering the mesh just prior to insertion in a trocar 40. A proximal end 32 of trap 30 tightly fits over the proximal portion of deployment rod 12, whereas a distal end 34 of trap 30 tightly fits over the distal portion of deployment rod 12. The middle portion 36 of finger trap 30 is enlarged and tightly fits over the mesh. In other words, the mesh is a reducible portion (i.e., it has a reducible perimeter) of mesh deployment device 10, and finger trap 30 applies a force on that reducible portion to reduce its perimeter.

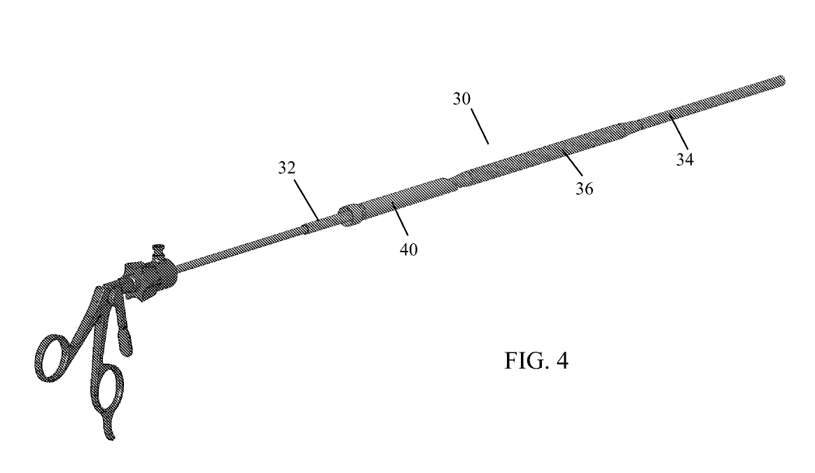

Reference is now made to FIG. 4, which illustrates finger trap 30 completely covering the mesh after insertion and pulling through trocar 40. Finger trap 30 acts a funnel for smooth insertion in trocar 40, thereby preventing bunching, snagging or unwinding before and during insertion via the trocar 40.

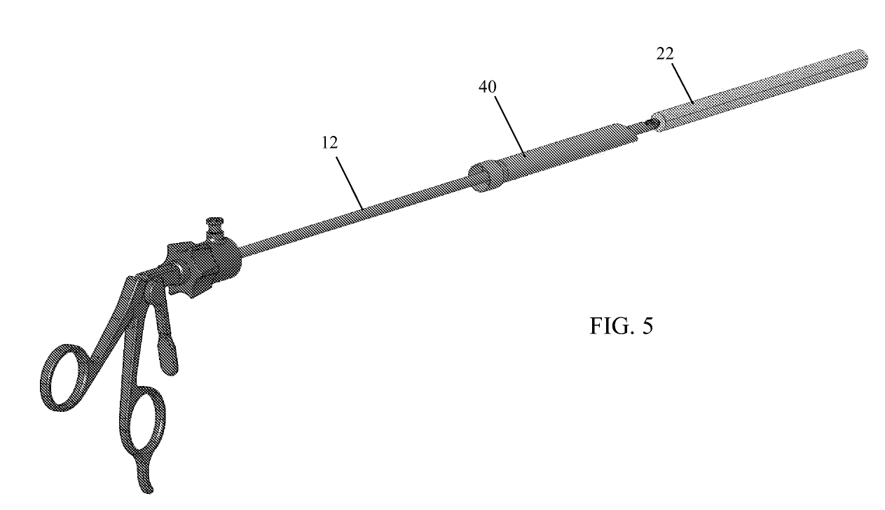

Reference is now made to FIG. 5, which illustrates the mesh deployment device 10 after insertion through trocar 40. Finger trap 30 has been removed by pulling it off mesh 22, either in the proximal or distal direction. A grasping tool (not shown) may be used to grasp and pull finger trap 30 off mesh 22. Device 10 may now be manipulated in a laparoscopic procedure for deploying the mesh.

In accordance with another embodiment of the invention, finger trap 30 may be removed from device 10 before insertion into trocar 40.

For example, mesh 22 is rolled onto roller portion 16. The rolled mesh 22 is then inserted into finger trap 30. As before, a narrow and relatively long remainder of the finger trap is left beyond the mesh. This remainder of the finger trap is inserted into a cannula or other funnel-like device (simply referred to as a funnel). The finger trap is then pulled together with the mesh along through the funnel. The funnel applies an inward radial force that squeezes and reduces the rolled mesh's diameter. The finger trap is then removed from off the mesh. The squeezing force applied on the mesh causes the mesh to take a set and remain with a reduced diameter. The mesh can then be easily introduced into and through trocar 40 without snagging and bunching.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. A method for insertion of a device in a trocar, comprising:
placing a finger trap over a reducible portion of a device that has a reducible perimeter, such that the finger trap applies a force on said reducible portion of the device to reduce its perimeter; and
inserting the device into a trocar.

2. The method according to claim 1, comprising inserting the device into the trocar with said finger trap fitted over said device, and afterwards removing said finger trap from said device.

3. The method according to claim 1, comprising first removing said finger trap from said device, and then inserting the device into the trocar.

4. The method according to claim 3, comprising leaving said finger trap on said reducible portion of said device for a sufficient time so that the force applied on said reducible portion of said device causes said reducible portion to take a set and remain with a reduced perimeter before insertion in said trocar.

5. The method according to claim 1, wherein a proximal end of said finger trap fits over a proximal portion of said device, a distal end of said finger trap fits over a distal portion of said device and a middle portion of said finger trap is enlarged and fits over said reducible portion of said device.

6. The method according to claim 1, wherein said reducible portion comprises a rolled mesh.

7. The method according to claim 1, wherein said device comprises a mesh deployment device that comprises a deployment rod having a handle at a proximal portion thereof and a roller portion at a distal portion thereof, and a rolled mesh detachably attached to said roller portion, wherein said reducible portion comprises said rolled mesh.

8. Apparatus for insertion of a device in a trocar, comprising:
a finger trap fitted over a reducible portion of a device that has a reducible perimeter, such that the finger trap applies a force on said reducible portion of the device to reduce its perimeter.

9. The apparatus according to claim 8, wherein a proximal end of said finger trap fits over a proximal portion of said device, a distal end of said finger trap fits over a distal portion of said device and a middle portion of said finger trap is enlarged and fits over said reducible portion of said device.

10. The apparatus according to claim 8, wherein said reducible portion comprises a rolled mesh.

11. The apparatus according to claim 8, wherein said device comprises a mesh deployment device that comprises a deployment rod having a handle at a proximal portion thereof and a roller portion at a distal portion thereof, and a rolled mesh detachably attached to said roller portion, wherein said reducible portion comprises said rolled mesh.
